(19) United States
(12) Patent Application Publication
(10) Pub. No.: US 2025/0154474 A1
(43) Pub. Date: May 15, 2025

(54) PRESERVATION SOLUTION FOR CRYOPRESERVATION OF ADIPOSE-DERIVED MESENCHYMAL STEM CELLS

(71) Applicant: Wuxi Cellular Biopharmaceutical Group Ltd., Wuxi, Jiangsu (CN)

(72) Inventors: Fei WANG, Wuxi, Jiangsu (CN); Dijun ZHAO, Wuxi, Jiangsu (CN); Qing YE, Wuxi, Jiangsu (CN); Dong XU, Wuxi, Jiangsu (CN); Luyi ZHANG, Wuxi, Jiangsu (CN); Junfeng WU, Wuxi, Jiangsu (CN); Shuqian YU, Wuxi, Jiangsu (CN); Li ZHANG, Wuxi, Jiangsu (CN)

(73) Assignee: Wuxi Cellular Biopharmaceutical Group Ltd., Wuxi, Jiangsu (CN)

(21) Appl. No.: 18/683,283

(22) PCT Filed: Aug. 11, 2022
(86) PCT No.: PCT/CN2022/111667

§ 371 Date: Sep. 9, 2024

(30) Foreign Application Priority Data
Aug. 13, 2021 (CN) 202110932618.1

(51) Int. Cl. C12N 5/00; C12N 5/0775

(52) U.S. Cl. CPC C12N 5/525; C12N 5/0667

(57) ABSTRACT

Provided is a preservation solution for low-temperature preservation of adipose-derived mesenchymal stem cells. The preservation solution contains a compound electrolyte injection, a glucose and sodium chloride injection, vitamins, and amino acids. The preservation solution can be used for preserving adipose-derived mesenchymal stem cells at a low temperature, so as to effectively maintain the viability of adipose-derived mesenchymal stem cells, and meet clinical requirements.

### TECHNICAL FIELD

The present invention relates to the field of biotechnology, and in particular, relates to a preservation solution for cryopreservation of adipose-derived mesenchymal stem cells.

### BACKGROUND

Adipose-derived mesenchymal stem cells (abbreviated as MSCs) are stem cells isolated from adipose tissue and having multidirectional differentiation potential. MSCs have the advantages of having relatively easily available starting materials, being capable of mass production, having low immunogenicity, and easily passing ethical review. As adult stem cells that have multidirectional differentiation potential, MSCs are currently the focus of most attention. They can be cultured and amplified in vitro, and can differentiate into a variety of tissue cells such as osteoblasts, chondrocytes, adipocytes, neural cells, and muscle cells.

Cell therapy requires large amounts of cells to meet clinical needs. Accordingly, in terms of cell production, a great number of cell repositories need to be established for cryopreservation, and the cells need to be resuscitated and washed with a cell preservation solution according to clinical requirements to prepare a final product, which is then delivered in a cold chain at 2° C. to 8° C. to a transportation center.

The cryopreserved cells are vulnerable and susceptible to disruption compared with fresh cells, and it takes multiple steps and a long time from resuscitating the cells to obtaining the final product. Without an appropriate cell preservation solution, it would be difficult to ensure the viability of MSCs, and the product quality and cell therapy result would be affected. In order that MSCs have high cell viability and good survivability so as to ensure their clinical performance, there is a need in the art to develop a preservation solution that is effective in preserving adipose-derived mesenchymal stem cells at a low temperature.

### SUMMARY

An object of the present invention is to provide a preservation solution for cryopreservation of cells, such as adipose-derived mesenchymal stem cells.

In a first aspect, the present invention provides a cell preservation solution, comprising:
a compound electrolyte;
glucose, and sodium chloride;
a vitamin; and
an amino acid.

In another preferred embodiment, the vitamin is selected from one or a combination of two or more of vitamin A, vitamin B, vitamin C, vitamin D, vitamin E, and vitamin K.

In another preferred embodiment, the amino acid is selected from one or a combination of two or more of glycine, alanine, valine, leucine, isoleucine, phenylalanine, proline, tryptophan, serine, tyrosine, cysteine, methionine, asparagine, glutamine, threonine, aspartic acid, glutamic acid, lysine, arginine, and histidine.

In another preferred embodiment, the vitamin is a water-soluble vitamin, and a 250 ml cell preservation solution comprises 1/48 to 1/24 of one tube of the water-soluble vitamin.

In another preferred embodiment, each tube of the water-soluble vitamin comprises: 2-5 mg of thiamine nitrate, 4-6 mg of riboflavin sodium phosphate, 30-50 mg of niacinamide, 4-6 mg of pyridoxine hydrochloride, 15-18 mg of sodium pantothenate, 100-135 mg of vitamin C sodium, 50-80 μg of biotin, 0.2-0.6 mg of folate, and 3-8 μg of vitamin B12.

In another preferred embodiment, each tube of the water-soluble vitamin comprises: 3.1 mg of thiamine nitrate, 4.9 mg of riboflavin sodium phosphate, 40 mg of niacinamide, 4.9 mg of pyridoxine hydrochloride, 16.5 mg of sodium pantothenate, 113 mg of vitamin C sodium, 60 μg of biotin, 0.4 mg of folate, and 5.0 μg of vitamin B12.

In another preferred embodiment, the preservation solution comprises one or a combination of two or more of the following:

0.06-0.13 mg/ml of thiamine nitrate, 0.1-0.2 mg/ml of riboflavin sodium phosphate, 0.8-1.6 mg/ml of niacinamide, 0.1-0.2 mg/ml of pyridoxine hydrochloride, 0.34-0.68 mg/ml of sodium pantothenate, 2.4-4.8 mg/ml of vitamin C sodium, 1.25-2.5 mg/ml of biotin, 0-0.16 mg/ml of folate, and 0.1-0.2 mg/ml of vitamin B12.

In another preferred embodiment, the water-soluble vitamin conforms to the standard requirements of “Water-soluble vitamins for injection” stipulated on page 44, volume V, Drug Standards (Part II), the Ministry of Health.

In another preferred embodiment, in the cell preservation solution, the volume of the amino acid accounts for 0.2% to 3%, preferably 0.2% to 2.5% of the total volume of the preservation solution.

In another preferred embodiment, each 250 ml of the amino acid comprises: 2-5 g of alanine, 1-4 g of arginine, 0.4-0.8 g of aspartic acid, 0.01-0.08 g of cystine, 0.8-1.5 g of glutamic acid, 1-2 g of glycine, 0.8-1.5 g of histidine, 1-2 g of leucine, 0.8-1.5 g of isoleucine, 0.8-1.5 g of methionine, and 1-2 g of phenylalanine.

In another preferred embodiment, the preservation solution comprises one or a combination of two or more of the following: 4.72-118 mg/ml of alanine, 2.36-94.4 mg/ml of arginine, 0.94-18.88 mg/ml of aspartic acid, 0.02-1.89 mg/ml of cystine, 1.89-35.4 mg/ml of glutamic acid, 2.36-47.2 mg/ml of glycine, 1.89-35.4 mg/ml of histidine, 2.36-94.4 mg/ml of leucine, 1.89-35.4 mg/ml of isoleucine, 1.89-35.4 mg/ml of methionine, and 2.36-94.4 mg/ml of phenylalanine.

In another preferred embodiment, each 250 ml of the amino acid comprises: 3.05 g of alanine, 2.10 g of arginine, 0.63 g of aspartic acid, 0.05 g of cystine, 1.05 g of glutamic acid, 1.48 g of glycine, 1.2 g of histidine, 1.48 g of leucine, 1.05 g of isoleucine, 1.05 g of methionine, and 1.48 g of phenylalanine.

In another preferred embodiment, each 1000 ml of a compound electrolyte injection comprises: 4-6 g of sodium chloride, 4-6 g of sodium gluconate, 3-5 g of sodium acetate, 0.2-0.5 g of potassium chloride, and 0.2-0.5 g of magnesium chloride.

In another preferred embodiment, the preservation solution comprises one or a combination of two or more of the following: 0.86-1.44 g/ml of sodium chloride, 0.86-1.44 g/ml of sodium gluconate, 0.64-1.44 g/ml of sodium acetate, 0.04-1.44 g/ml of potassium chloride, and 0.04-1.44 g/ml of magnesium chloride.

In another preferred embodiment, each 1000 ml of the compound electrolyte injection comprises: 5.26 g of sodium chloride, 5.02 g of sodium gluconate, 3.68 g of sodium acetate, 0.37 g of potassium chloride, and 0.30 g of magnesium chloride.

In another preferred embodiment, the volume ratio of the compound electrolyte to the preservation solution is 80% to 98% (V/V), preferably 85% to 96% (V/V).

In another preferred embodiment, each 100 ml of a glucose-sodium chloride injection comprises 4-6 g of glucose and 0.85-0.95 g of sodium chloride.

In another preferred embodiment, the volume of the glucose-sodium chloride injection accounts for 1% to 15% of the total volume of the preservation solution.

In another preferred embodiment, the glucose-sodium chloride injection comprises 5% of glucose and 0.9% of sodium chloride.

In another preferred embodiment, the volume of the glucose-sodium chloride injection accounts for 2% to 14%, preferably 3% to 12% of the total volume of the preservation solution.

In another preferred embodiment, the preservation solution comprises one or a combination of two of the following: 0.32-1.8 g/ml of glucose; and 0.07-0.28 g/ml of sodium chloride.

The preservation composition can be used to preserve/store any suitable cells at a low temperature.

In some embodiments, the cells are mammalian cells. In some embodiments, the cells are human, procine, canine, equine, or bovine cells. In some embodiments, the cells include stem cells or progenitor cells. The cells may be hematopoietic cells, such as lymphocytes, including T lymphocytes and B lymphocytes. The cells may be immunocytes.

In some embodiments, the preservation composition of the present invention is used to preserve stem cells or progenitor cells. Stem cells are undifferentiated cells having the ability to self-renew and produce differentiated progenies (see Morrison et al., (1997) Cell, 88:287-298). In mammals, there exists two broad types of stem cells: embryonic stem cells and adult stem cells, and they are present in various tissues.

Stem cells may be bone marrow-derived stem cells (BMSCs), adipose-derived stem cells (ADSCs), neural stem cells (NSCs), blood stem cells, or hematopoietic stem cells. Stem cells may also be derived from umbilical cord blood. Stem cells may be produced by somatic nuclear transplantation or dedifferentiation.

Stem cells include, but are not limited to, blood stem cells, adipose stem cells, bone marrow mesenchymal stem cells, mesenchymal stem cells, neural stem cells (NSCs), skin stem cells, endothelial stem cells, liver stem cells, pancreatic stem cells, intestinal epithelial stem cells, or reproductive stem cells. In some embodiments, mesenchymal stem cells are isolated from mesodermal organs, such as bone marrow, umbilical cord blood, and adipose tissue.

In some embodiments, the stem cells are induced pluripotent stem cells (IPS cells or iPSCs). iPSCs refer to a class of pluripotent stem cells that are artificially produced from non-pluripotent cells, and are typically adult stem cells, or terminally differentiated cells, such as fibroblasts, hematopoietic cells, muscle cells, neurons, epidermal cells, etc.

The preservation composition of the present invention can be used to preserve any stem cells having differentiation potential, including, but not limited to, embryonic stem cells, induced high-performance stem cells, cancer stem cells, and tissue stem cells. Tissue stem cells include, but are not limited to, mesenchymal stem cells, hematopoietic stem cells, breast stem cells, neural stem cells, small intestine stem cells, skin stem cells, umbilical cord blood stem cells, corneal limbal stem cells, hair follicle stem cells, adipose tissue-derived stem cells, bone marrow stem cells, corneal stem cells, and ovarian stem cells. Stem cells used for producing exosomes can be selected from embryonic stem cells, induced pluripotent stem cells, cancer stem cells, mesenchymal stem cells, hematopoietic stem cells, breast stem cells, neural stem cells, small intestine stem cells, skin stem cells, umbilical cord blood stem cells, corneal limbal stem cells, hair follicle stem cells, adipose tissue-derived stem cells, bone marrow stem cells, corneal stem cells, and ovarian stem cells.

In a second aspect, the present invention provides a mixture of cells (such as adipose-derived mesenchymal stem cells), the mixture of cells (such as adipose-derived mesenchymal stem cells) comprising cells (such as adipose-derived mesenchymal stem cells) and the preservation solution according to the first aspect.

In another preferred embodiment, the cells (such as adipose-derived mesenchymal stem cells) are resuscitated cells (such as adipose-derived mesenchymal stem cells).

In another preferred embodiment, the ratio of the cells (such as adipose-derived mesenchymal stem cells) to the preservation solution is 1-5×10⁷ cells: 1-3 mL preservation solution.

In some embodiments, the cells are present in the preservation composition at a concentration of about 10⁵ cells/ml to about 10⁷ cells/ml.

In another preferred embodiment, in the adipose-derived mesenchymal stem cells, the cells that account for 95% or more of the total number of cells have surface antigen CD90,
the cells that account for 95% or more of the total number of cells have surface antigen CD73;
the cells that account for 95% or more of the total number of cells have surface antigen CD105;
the cells having surface antigen HLA-DR account for 0% to 0.5% of the total number of cells;
the cells having surface antigen CD45 account for 0% to 5% of the total number of cells; and/or
the cells having surface antigen CD14 account for 0% to 5% of the total number of cells.

In a third aspect, the present invention provides a method for cryopreservation of cells, the method comprising the steps of suspending the cells in the cell preservation solution according to the first aspect, and then preserving the cells at a low temperature.

In another preferred embodiment, the low temperature refers to 2° C. to 8° C.

In another preferred embodiment, the low temperature is 0° C. to 10° C.

In another preferred embodiment, the low temperature is 3.5° C. to 4.5° C.

In another preferred embodiment, the cells are adipose-derived mesenchymal stem cells.

In another preferred embodiment, the adipose-derived mesenchymal stem cells are resuscitated adipose-derived mesenchymal stem cells.

In a fourth aspect, present invention provides a repository of adipose-derived mesenchymal stem cells, the repository of adipose-derived mesenchymal stem cells comprising the mixture of adipose-derived mesenchymal stem cells according to the second aspect.

In a fifth aspect, the present invention provides use of the preservation solution according to the first aspect in the preservation of adipose-derived mesenchymal stem cells, the establishment of a repository of adipose-derived mesenchymal stem cells, the maintenance of the viability of adipose-derived mesenchymal stem cells, or the retainment of the surface markers of adipose-derived mesenchymal stem cells.

In another preferred embodiment, the preservation refers to preservation at a low temperature, or cryopreservation, wherein the low temperature refers to 2° C. to 8° C.

In another preferred embodiment, the adipose-derived mesenchymal stem cells are resuscitated adipose-derived mesenchymal stem cells.

The preservation solution for preserving adipose-derived mesenchymal stem cells provided in the present invention comprises a compound electrolyte added with an amino acid, a vitamin, as well as glucose and sodium chloride. The cells preserved with the preservation solution at a low temperature condition of 2° C. to 8° C. for 72 hours can reach a cell viability of 70% or higher, which can meet clinical requirements.

In some embodiments, the cells have at least 70% or at least 80% viability after storage or preservation.

The present invention further provides a kit comprising the preservation composition of the present invention.

It is to be understood that, within the scope of the present invention, the above technical features of the present invention and technical features specifically described below (such as examples) can be combined with each other to form new or preferred technical solutions. The features disclosed in the description may be substituted by any alternative features that provide an identical, equivalent, or similar purpose. Due to limited space, details are not described herein.

### BRIEF DESCRIPTION OF THE DRAWINGS

FIG. 1 shows images of cell morphology of cells having been preserved for 0 h, wherein A: cell morphology of cells having been preserved in CE01KS for 0 h, on day 1 after inoculation; B: cell morphology of cells having been preserved in CE01KS for 0 h, on day 3 after inoculation; C: cell morphology of cells having been preserved in physiological saline for 0 h, on day 1 after inoculation; and D: cell morphology of cells having been preserved in physiological saline for 0 h, on day 3 after inoculation.

FIG. 2 shows images of cell morphology of cells having been preserved for 24 h, wherein E: cell morphology of cells having been preserved in CE01KS at a low temperature (such as at 2° C. to 8° C. in a refrigerator) for 24 h, on day 1 after inoculation; F: cell morphology of cells having been preserved in CE01KS in a refrigerator for 24 h, on day 4 after inoculation; G: cell morphology of cells having been preserved in physiological saline in a refrigerator for 24 h, on day 1 after inoculation; and H: cell morphology of cells having been preserved in physiological saline in a refrigerator for 24 h, on day 4 after inoculation.

FIG. 3 shows images of cell morphology of cells having been preserved for 48 h, wherein I: cell morphology of cells having been preserved in CE01KS in a refrigerator for 48 h, on day 1 after inoculation; K: cell morphology of cells having been preserved in CE01KS in a refrigerator for 48 h, on day 6 after inoculation; J: cell morphology of cells having been preserved in physiological saline in a refrigerator for 48 b, on day 1 after inoculation; and L: cell morphology of cells having been preserved in physiological saline in a refrigerator for 48 h, on day 6 after inoculation.

FIG. 4 shows apoptosis plots of cells having been preserved for 0 b, and for 24 h, 48 h, and 72 h in a refrigerator.

FIG. 5 shows the change in surface markers of cells having been preserved for 0 h, and for 24 h and 48 h in a refrigerator.

FIG. 6 shows flow plots of surface markers of cells having been preserved for 0 h.

FIG. 7 shows flow plots of surface markers of cells having been preserved in a refrigerator for 24 h.

FIG. 8 shows flow plots of surface markers of cells having been preserved in a refrigerator for 48 h.

### DETAILED DESCRIPTION OF THE EMBODIMENTS

On the basis of long-term and in-depth research, the present inventors developed a preservation solution for preserving adipose-derived mesenchymal stem cells, particularly resuscitated adipose-derived mesenchymal stem cells, comprising a compound electrolyte added with an amino acid, a vitamin, as well as glucose and sodium chloride. The cells preserved with the preservation solution at a low temperature condition of 2° C. to 8° C. for 72 hours had a cell viability of more than 70%. On this basis, the inventors have completed the present invention.

In some embodiments, the preservation composition may be free of, or comprise, sodium chloride in the following concentration range: about 0.5 g/ml to about 3 g/ml, about 0.5 g/ml to about 2.8 g/ml, about 0.5 g/ml to about 2.5 g/ml, about 0.5 g/ml to about 2.2 g/ml, about 0.5 g/ml to about 2 g/ml, about 0.5 g/ml to about 1.8 g/ml, about 0.5 g/ml to about 1.5 g/ml, about 0.5 g/ml to about 1.3 g/ml, about 0.5 g/ml to about 1 g/ml, about 0.8 g/ml to about 3 g/ml, about 0.8 g/ml to about 2.8 g/ml, about 0.8 g/ml to about 2.5 g/ml, about 0.8 g/ml to about 2.2 g/ml, about 0.8 g/ml to about 2 g/ml, about 0.8 g/ml to about 1.8 g/ml, about 0.8 g/ml to about 1.5 g/ml, about 0.8 g/ml to about 1.3 g/ml, or about 0.8 g/ml to about 1 g/ml.

In some embodiments, the preservation composition may be free of, or comprise, glucose in the following concentration range: about 0.1 g/ml to about 3 g/ml, about 0.1 g/ml to about 2.8 g/ml, about 0.1 g/ml to about 2.5 g/ml, about 0.1 g/ml to about 2.2 g/ml, about 0.1 g/ml to about 2 g/ml, about 0.1 g/ml to about 1.8 g/ml, about 0.1 g/ml to about 1.5 g/ml, about 0.1 g/ml to about 1.3 g/ml, about 0.1 g/ml to about 1 g/ml, about 0.3 g/ml to about 3 g/ml, about 0.3 g/ml to about 2.8 g/ml, about 0.3 g/ml to about 2.5 g/ml, about 0.3 g/ml to about 2.2 g/ml, about 0.3 g/ml to about 2 g/ml, about 0.3 g/ml to about 1.8 g/ml, about 0.3 g/ml to about 1.5 g/ml, about 0.3 g/ml to about 1.3 g/ml, about 0.3 g/ml to about 1 g/ml, about 0.5 g/ml to about 3 g/ml, about 0.5 g/ml to about 2.8 g/ml, about 0.5 g/ml to about 2.5 g/ml, about 0.5 g/ml to about 2.2 g/ml, about 0.5 g/ml to about 2 g/ml, about 0.5 g/ml to about 1.8 g/ml, about 0.5 g/ml to about 1.5 g/ml, about 0.5 g/ml to about 1.3 g/ml, about 0.5 g/ml to about 1 g/ml, about 0.8 g/ml to about 3 g/ml, about 0.8 g/ml to about 2.8 g/ml, about 0.8 g/ml to about 2.5 g/ml, about 0.8 g/ml to about 2.2 g/ml, about 0.8 g/ml to about 2 g/ml, about 0.8 g/ml to about 1.8 g/ml, about 0.8 g/ml to about 1.5 g/ml, about 0.8 g/ml to about 1.3 g/ml, or about 0.8 g/ml to about 1 g/ml.

In some embodiments, the preservation composition may be free of, or comprise, sodium gluconate in the following concentration range: about 0.5 g/ml to about 3 g/ml, about 0.5 g/ml to about 2.8 g/ml, about 0.5 g/ml to about 2.5 g/ml, about 0.5 g/ml to about 2.2 g/ml, about 0.5 g/ml to about 2 g/ml, about 0.5 g/ml to about 1.8 g/ml, about 0.5 g/ml to about 1.5 g/ml, about 0.5 g/ml to about 1.3 g/ml, about 0.5 g/ml to about 1 g/ml, about 0.8 g/ml to about 3 g/ml, about 0.8 g/ml to about 2.8 g/ml, about 0.8 g/ml to about 2.5 g/ml, about 0.8 g/ml to about 2.2 g/ml, about 0.8 g/ml to about 2 g/ml, about 0.8 g/ml to about 1.8 g/ml, about 0.8 g/ml to about 1.5 g/ml, about 0.8 g/ml to about 1.3 g/ml, or about 0.8 g/ml to about 1 g/ml.

In some embodiments, the preservation composition may be free of, or comprise, sodium acetate in the following concentration range: about 0.3 g/ml to about 3 g/ml, about 0.3 g/ml to about 2.8 g/ml, about 0.3 g/ml to about 2.5 g/ml, about 0.3 g/ml to about 2.2 g/ml, about 0.3 g/ml to about 2 g/ml, about 0.3 g/ml to about 1.8 g/ml, about 0.3 g/ml to about 1.5 g/ml, about 0.3 g/ml to about 1.3 g/ml, about 0.3 g/ml to about 1 g/ml, about 0.5 g/ml to about 3 g/ml, about 0.5 g/ml to about 2.8 g/ml, about 0.5 g/ml to about 2.5 g/ml, about 0.5 g/ml to about 2.2 g/ml, about 0.5 g/ml to about 2 g/ml, about 0.5 g/ml to about 1.8 g/ml, about 0.5 g/ml to about 1.5 g/ml, about 0.5 g/ml to about 1.3 g/ml, about 0.5 g/ml to about 1 g/ml, about 0.8 g/ml to about 3 g/ml, about 0.8 g/ml to about 2.8 g/ml, about 0.8 g/ml to about 2.5 g/ml, about 0.8 g/ml to about 2.2 g/ml, about 0.8 g/ml to about 2 g/ml, about 0.8 g/ml to about 1.8 g/ml, about 0.8 g/ml to about 1.5 g/ml, about 0.8 g/ml to about 1.3 g/ml, or about 0.8 g/ml to about 1 g/ml.

In some embodiments, the preservation composition may be free of, or comprise, potassium chloride in the following concentration range: about 0.01 g/ml to about 3 g/ml, about 0.01 g/ml to about 2.8 g/ml, about 0.01 g/ml to about 2.5 g/ml, about 0.01 g/ml to about 2.2 g/ml, about 0.01 g/ml to about 2 g/ml, about 0.01 g/ml to about 1.8 g/ml, about 0.01 g/ml to about 1.5 g/ml, about 0.01 g/ml to about 1.3 g/ml, about 0.01 g/ml to about 1 g/ml, about 0.03 g/ml to about 3 g/ml, about 0.03 g/ml to about 2.8 g/ml, about 0.03 g/ml to about 2.5 g/ml, about 0.03 g/ml to about 2.2 g/ml, about 0.03 g/ml to about 2 g/ml, about 0.03 g/ml to about 1.8 g/ml, about 0.03 g/ml to about 1.5 g/ml, about 0.03 g/ml to about 1.3 g/ml, about 0.03 g/ml to about 1 g/ml, about 0.04 g/ml to about 3 g/ml, about 0.04 g/ml to about 2.8 g/ml, about 0.04 g/ml to about 2.5 g/ml, about 0.04 g/ml to about 2.2 g/ml, about 0.04 g/ml to about 2 g/ml, about 0.04 g/ml to about 1.8 g/ml, about 0.04 g/ml to about 1.5 g/ml, about 0.04 g/ml to about 1.3 g/ml, about 0.04 g/ml to about 1 g/ml, about 0.1 g/ml to about 3 g/ml, about 0.1 g/ml to about 2.8 g/ml, about 0.1 g/ml to about 2.5 g/ml, about 0.1 g/ml to about 2.2 g/ml, about 0.1 g/ml to about 2 g/ml, about 0.1 g/ml to about 1.8 g/ml, about 0.1 g/ml to about 1.5 g/ml, about 0.1 g/ml to about 1.3 g/ml, about 0.1 g/ml to about 1 g/ml, about 0.3 g/ml to about 3 g/ml, about 0.3 g/ml to about 2.8 g/ml, about 0.3 g/ml to about 2.5 g/ml, about 0.3 g/ml to about 2.2 g/ml, about 0.3 g/ml to about 2 g/ml, about 0.3 g/ml to about 1.8 g/ml, about 0.3 g/ml to about 1.5 g/ml, about 0.3 g/ml to about 1.3 g/ml, about 0.3 g/ml to about 1 g/ml, about 0.5 g/ml to about 3 g/ml, about 0.5 g/ml to about 2.8 g/ml, about 0.5 g/ml to about 2.5 g/ml, about 0.5 g/ml to about 2.2 g/ml, about 0.5 g/ml to about 2 g/ml, about 0.5 g/ml to about 1.8 g/ml, about 0.5 g/ml to about 1.5 g/ml, about 0.5 g/ml to about 1.3 g/ml, about 0.5 g/ml to about 1 g/ml, about 0.8 g/ml to about 3 g/ml, about 0.8 g/ml to about 2.8 g/ml, about 0.8 g/ml to about 2.5 g/ml, about 0.8 g/ml to about 2.2 g/ml, about 0.8 g/ml to about 2 g/ml, about 0.8 g/ml to about 1.8 g/ml, about 0.8 g/ml to about 1.5 g/ml, about 0.8 g/ml to about 1.3 g/ml, or about 0.8 g/ml to about 1 g/ml.

In some embodiments, the preservation composition may be free of, or comprise, magnesium chloride in the following concentration range: about 0.01 g/ml to about 3 g/ml, about 0.01 g/ml to about 2.8 g/ml, about 0.01 g/ml to about 2.5 g/ml, about 0.01 g/ml to about 2.2 g/ml, about 0.01 g/ml to about 2 g/ml, about 0.01 g/ml to about 1.8 g/ml, about 0.01 g/ml to about 1.5 g/ml, about 0.01 g/ml to about 1.3 g/ml, about 0.01 g/ml to about 1 g/ml, about 0.03 g/ml to about 3 g/ml, about 0.03 g/ml to about 2.8 g/ml, about 0.03 g/ml to about 2.5 g/ml, about 0.03 g/ml to about 2.2 g/ml, about 0.03 g/ml to about 2 g/ml, about 0.03 g/ml to about 1.8 g/ml, about 0.03 g/ml to about 1.5 g/ml, about 0.03 g/ml to about 1.3 g/ml, about 0.03 g/ml to about 1 g/ml, about 0.04 g/ml to about 3 g/ml, about 0.04 g/ml to about 2.8 g/ml, about 0.04 g/ml to about 2.5 g/ml, about 0.04 g/ml to about 2.2 g/ml, about 0.04 g/ml to about 2 g/ml, about 0.04 g/ml to about 1.8 g/ml, about 0.04 g/ml to about 1.5 g/ml, about 0.04 g/ml to about 1.3 g/ml, about 0.04 g/ml to about 1 g/ml, about 0.1 g/ml to about 3 g/ml, about 0.1 g/ml to about 2.8 g/ml, about 0.1 g/ml to about 2.5 g/ml, about 0.1 g/ml to about 2.2 g/ml, about 0.1 g/ml to about 2 g/ml, about 0.1 g/ml to about 1.8 g/ml, about 0.1 g/ml to about 1.5 g/ml, about 0.1 g/ml to about 1.3 g/ml, about 0.1 g/ml to about 1 g/ml, about 0.3 g/ml to about 3 g/ml, about 0.3 g/ml to about 2.8 g/ml, about 0.3 g/ml to about 2.5 g/ml, about 0.3 g/ml to about 2.2 g/ml, about 0.3 g/ml to about 2 g/ml, about 0.3 g/ml to about 1.8 g/ml, about 0.3 g/ml to about 1.5 g/ml, about 0.3 g/ml to about 1.3 g/ml, about 0.3 g/ml to about 1 g/ml, about 0.5 g/ml to about 3 g/ml, about 0.5 g/ml to about 2.8 g/ml, about 0.5 g/ml to about 2.5 g/ml, about 0.5 g/ml to about 2.2 g/ml, about 0.5 g/ml to about 2 g/ml, about 0.5 g/ml to about 1.8 g/ml, about 0.5 g/ml to about 1.5 g/ml, about 0.5 g/ml to about 1.3 g/ml, about 0.5 g/ml to about 1 g/ml, about 0.8 g/ml to about 3 g/ml, about 0.8 g/ml to about 2.8 g/ml, about 0.8 g/ml to about 2.5 g/ml, about 0.8 g/ml to about 2.2 g/ml, about 0.8 g/ml to about 2 g/ml, about 0.8 g/ml to about 1.8 g/ml, about 0.8 g/ml to about 1.5 g/ml, about 0.8 g/ml to about 1.3 g/ml, or about 0.8 g/ml to about 1 g/ml.

In some embodiments, the preservation composition may be free of, or comprise, thiamine nitrate in the following concentration range: about 0.01 mg/ml to about 1.5 mg/ml, about 0.01 mg/ml to about 1.3 mg/ml, about 0.01 mg/ml to about 1 mg/ml, about 0.01 mg/ml to about 0.8 mg/ml, about 0.01 mg/ml to about 0.5 mg/ml, about 0.01 mg/ml to about 0.3 mg/ml, about 0.01 mg/ml to about 0.2 mg/ml, about 0.02 mg/ml to about 1.5 mg/ml, about 0.02 mg/ml to about 1.3 mg/ml, about 0.02 mg/ml to about 1 mg/ml, about 0.02 mg/ml to about 0.8 mg/ml, about 0.02 mg/ml to about 0.5 mg/ml, about 0.02 mg/ml to about 0.3 mg/ml, about 0.02 mg/ml to about 0.2 mg/ml, about 0.03 mg/ml to about 1.5 mg/ml, about 0.03 mg/ml to about 1.3 mg/ml, about 0.03 mg/ml to about 1 mg/ml, about 0.03 mg/ml to about 0.8 mg/ml, about 0.03 mg/ml to about 0.5 mg/ml, about 0.03 mg/ml to about 0.3 mg/ml, about 0.03 mg/ml to about 0.2 mg/ml, about 0.04 mg/ml to about 1.5 mg/ml, about 0.04 mg/ml to about 1.3 mg/ml, about 0.04 mg/ml to about 1 mg/ml, about 0.04 mg/ml to about 0.8 mg/ml, about 0.04 mg/ml to about 0.5 mg/ml, about 0.04 mg/ml to about 0.3 mg/ml, about 0.04 mg/ml to about 0.2 mg/ml, about 0.05 mg/ml to about 1.5 mg/ml, about 0.05 mg/ml to about 1.3 mg/ml, about 0.05 mg/ml to about 1 mg/ml, about 0.05 mg/ml to about 0.8 mg/ml, about 0.05 mg/ml to about 0.5 mg/ml, about 0.05 mg/ml to about 0.3 mg/ml, or about 0.05 mg/ml to about 0.2 mg/ml.

In some embodiments, the preservation composition may be free of, or comprise, riboflavin sodium phosphate in the following concentration range: about 0.01 mg/ml to about 1.5 mg/ml, about 0.01 mg/ml to about 1.3 mg/ml, about 0.01 mg/ml to about 1 mg/ml, about 0.01 mg/ml to about 0.8 mg/ml, about 0.01 mg/ml to about 0.5 mg/ml, about 0.01 mg/ml to about 0.3 mg/ml, about 0.01 mg/ml to about 0.2 mg/ml, about 0.03 mg/ml to about 1.5 mg/ml, about 0.03 mg/ml to about 1.3 mg/ml, about 0.03 mg/ml to about 1 mg/ml, about 0.03 mg/ml to about 0.8 mg/ml, about 0.03 mg/ml to about 0.5 mg/ml, about 0.03 mg/ml to about 0.3 mg/ml, about 0.03 mg/ml to about 0.2 mg/ml, about 0.05 mg/ml to about 1.5 mg/ml, about 0.05 mg/ml to about 1.3 mg/ml, about 0.05 mg/ml to about 1 mg/ml, about 0.05 mg/ml to about 0.8 mg/ml, about 0.05 mg/ml to about 0.5 mg/ml, about 0.05 mg/ml to about 0.3 mg/ml, about 0.05 mg/ml to about 0.2 mg/ml, about 0.08 mg/ml to about 1.5 mg/ml, about 0.08 mg/ml to about 1.3 mg/ml, about 0.08 mg/ml to about 1 mg/ml, about 0.08 mg/ml to about 0.8 mg/ml, about 0.08 mg/ml to about 0.5 mg/ml, about 0.08 mg/ml to about 0.3 mg/ml, about 0.08 mg/ml to about 0.2 mg/ml, about 0.1 mg/ml to about 1.5 mg/ml, about 0.1 mg/ml to about 1.3 mg/ml, about 0.1 mg/ml to about 1 mg/ml, about 0.1 mg/ml to about 0.8 mg/ml, about 0.1 mg/ml to about 0.5 mg/ml, about 0.1 mg/ml to about 0.3 mg/ml, or about 0.1 mg/ml to about 0.2 mg/ml.

In some embodiments, the preservation composition may be free of, or comprise, niacinamide in the following concentration range: about 0.1 mg/ml to about 3 mg/ml, about 0.1 mg/ml to about 2.5 mg/ml, about 0.1 mg/ml to about 2 mg/ml, about 0.1 mg/ml to about 1.5 mg/ml, about 0.1 mg/ml to about 1.3 mg/ml, about 0.1 mg/ml to about 1 mg/ml, about 0.1 mg/ml to about 0.8 mg/ml, about 0.3 mg/ml to about 3 mg/ml, about 0.3 mg/ml to about 2.5 mg/ml, about 0.3 mg/ml to about 2 mg/ml, about 0.3 mg/ml to about 1.5 mg/ml, about 0.3 mg/ml to about 1.3 mg/ml, about 0.3 mg/ml to about 1 mg/ml, about 0.3 mg/ml to about 0.8 mg/ml, about 0.5 mg/ml to about 3 mg/ml, about 0.5 mg/ml to about 2.5 mg/ml, about 0.5 mg/ml to about 2 mg/ml, about 0.5 mg/ml to about 1.5 mg/ml, about 0.5 mg/ml to about 1.3 mg/ml, about 0.5 mg/ml to about 1 mg/ml, about 0.5 mg/ml to about 0.8 mg/ml, about 0.8 mg/ml to about 3 mg/ml, about 0.8 mg/ml to about 2.5 mg/ml, about 0.8 mg/ml to about 2 mg/ml, about 0.8 mg/ml to about 1.5 mg/ml, about 0.8 mg/ml to about 1.3 mg/ml, or about 0.8 mg/ml to about 1 mg/ml.

In some embodiments, the preservation composition may be free of, or comprise, pyridoxine hydrochloride in the following concentration range: about 0.01 mg/ml to about 1.5 mg/ml, about 0.01 mg/ml to about 1.3 mg/ml, about 0.01 mg/ml to about 1 mg/ml, about 0.01 mg/ml to about 0.8 mg/ml, about 0.01 mg/ml to about 0.5 mg/ml, about 0.01 mg/ml to about 0.3 mg/ml, about 0.01 mg/ml to about 0.2 mg/ml, about 0.03 mg/ml to about 1.5 mg/ml, about 0.03 mg/ml to about 1.3 mg/ml, about 0.03 mg/ml to about 1 mg/ml, about 0.03 mg/ml to about 0.8 mg/ml, about 0.03 mg/ml to about 0.5 mg/ml, about 0.03 mg/ml to about 0.3 mg/ml, about 0.03 mg/ml to about 0.2 mg/ml, about 0.05 mg/ml to about 1.5 mg/ml, about 0.05 mg/ml to about 1.3 mg/ml, about 0.05 mg/ml to about 1 mg/ml, about 0.05 mg/ml to about 0.8 mg/ml, about 0.05 mg/ml to about 0.5 mg/ml, about 0.05 mg/ml to about 0.3 mg/ml, about 0.05 mg/ml to about 0.2 mg/ml, about 0.08 mg/ml to about 1.5 mg/ml, about 0.08 mg/ml to about 1.3 mg/ml, about 0.08 mg/ml to about 1 mg/ml, about 0.08 mg/ml to about 0.8 mg/ml, about 0.08 mg/ml to about 0.5 mg/ml, about 0.08 mg/ml to about 0.3 mg/ml, about 0.08 mg/ml to about 0.2 mg/ml, about 0.1 mg/ml to about 1.5 mg/ml, about 0.1 mg/ml to about 1.3 mg/ml, about 0.1 mg/ml to about 1 mg/ml, about 0.1 mg/ml to about 0.8 mg/ml, about 0.1 mg/ml to about 0.5 mg/ml, about 0.1 mg/ml to about 0.3 mg/ml, or about 0.1 mg/ml to about 0.2 mg/ml.

In some embodiments, the preservation composition may be free of, or comprise, sodium pantothenate in the following concentration range: about 0.01 mg/ml to about 1.5 mg/ml, about 0.01 mg/ml to about 1.3 mg/ml, about 0.01 mg/ml to about 1 mg/ml, about 0.01 mg/ml to about 0.8 mg/ml, about 0.01 mg/ml to about 0.7 mg/ml, about 0.01 mg/ml to about 0.6 mg/ml, about 0.01 mg/ml to about 0.5 mg/ml, about 0.03 mg/ml to about 1.5 mg/ml, about 0.03 mg/ml to about 1.3 mg/ml, about 0.03 mg/ml to about 1 mg/ml, about 0.03 mg/ml to about 0.8 mg/ml, about 0.03 mg/ml to about 0.7 mg/ml, about 0.03 mg/ml to about 0.6 mg/ml, about 0.03 mg/ml to about 0.5 mg/ml, about 0.05 mg/ml to about 1.5 mg/ml, about 0.05 mg/ml to about 1.3 mg/ml, about 0.05 mg/ml to about 1 mg/ml, about 0.05 mg/ml to about 0.8 mg/ml, about 0.05 mg/ml to about 0.7 mg/ml, about 0.05 mg/ml to about 0.6 mg/ml, about 0.08 mg/ml to about 1.5 mg/ml, about 0.08 mg/ml to about 1.3 mg/ml, about 0.08 mg/ml to about 1 mg/ml, about 0.08 mg/ml to about 0.8 mg/ml, about 0.08 mg/ml to about 0.7 mg/ml, about 0.08 mg/ml to about 0.6 mg/ml, about 0.08 mg/ml to about 0.5 mg/ml, about 0.1 mg/ml to about 1.5 mg/ml, about 0.1 mg/ml to about 1.3 mg/ml, about 0.1 mg/ml to about 1 mg/ml, about 0.1 mg/ml to about 0.8 mg/ml, about 0.1 mg/ml to about 0.7 mg/ml, about 0.1 mg/ml to about 0.6 mg/ml, about 0.1 mg/ml to about 0.5 mg/ml, about 0.2 mg/ml to about 1.5 mg/ml, about 0.2 mg/ml to about 1.3 mg/ml, about 0.2 mg/ml to about 1 mg/ml, about 0.2 mg/ml to about 0.8 mg/ml, about 0.2 mg/ml to about 0.7 mg/ml, about 0.2 mg/ml to about 0.6 mg/ml, about 0.2 mg/ml to about 0.5 mg/ml, about 0.3 mg/ml to about 1.5 mg/ml, about 0.3 mg/ml to about 1.3 mg/ml, about 0.3 mg/ml to about 1 mg/ml, about 0.3 mg/ml to about 0.8 mg/ml, about 0.3 mg/ml to about 0.7 mg/ml, about 0.3 mg/ml to about 0.6 mg/ml, or about 0.3 mg/ml to about 0.5 mg/ml.

In some embodiments, the preservation composition may be free of, or comprise, vitamin C sodium in the following concentration range: about 1 mg/ml to about 10 mg/ml, about 1 mg/ml to about 8 mg/ml, about 1 mg/ml to about 5 mg/ml, about 1 mg/ml to about 4 mg/ml, about 1 mg/ml to about 3 mg/ml, about 1.5 mg/ml to about 10 mg/ml, about 1.5 mg/ml to about 8 mg/ml, about 1.5 mg/ml to about 5 mg/ml, about 1.5 mg/ml to about 4 mg/ml, about 1.5 mg/ml to about 3 mg/ml, about 2 mg/ml to about 10 mg/ml, about 2 mg/ml to about 8 mg/ml, about 2 mg/ml to about 5 mg/ml, about 2 mg/ml to about 4 mg/ml, about 2 mg/ml to about 3 mg/ml, about 2.5 mg/ml to about 10 mg/ml, about 2.5 mg/ml to about 8 mg/ml, about 2.5 mg/ml to about 5 mg/ml, about 2.5 mg/ml to about 4 mg/ml, or about 2.5 mg/ml to about 3 mg/ml.

In some embodiments, the preservation composition may be free of, or comprise, biotin in the following concentration range: about 0.5 mg/ml to about 10 mg/ml, about 0.5 mg/ml to about 8 mg/ml, about 0.5 mg/ml to about 5 mg/ml, about 0.5 mg/ml to about 4 mg/ml, about 0.5 mg/ml to about 3 mg/ml, about 0.8 mg/ml to about 10 mg/ml, about 0.8 mg/ml to about 8 mg/ml, about 0.8 mg/ml to about 5 mg/ml, about 0.8 mg/ml to about 4 mg/ml, about 0.8 mg/ml to about 3 mg/ml, about 1 mg/ml to about 10 mg/ml, about 1 mg/ml to about 8 mg/ml, about 1 mg/ml to about 5 mg/ml, about 1 mg/ml to about 4 mg/ml, about 1 mg/ml to about 3 mg/ml, about 1.5 mg/ml to about 10 mg/ml, about 1.5 mg/ml to about 8 mg/ml, about 1.5 mg/ml to about 5 mg/ml, about 1.5 mg/ml to about 4 mg/ml, about 1.5 mg/ml to about 3 mg/ml, about 2 mg/ml to about 10 mg/ml, about 2 mg/ml to about 8 mg/ml, about 2 mg/ml to about 5 mg/ml, about 2 mg/ml to about 4 mg/ml, about 2 mg/ml to about 3 mg/ml, about 2.5 mg/ml to about 10 mg/ml, about 2.5 mg/ml to about 8 mg/ml, about 2.5 mg/ml to about 5 mg/ml, about 2.5 mg/ml to about 4 mg/ml, or about 2.5 mg/ml to about 3 mg/ml.

In some embodiments, the preservation composition may be free of, or comprise, folate in the following concentration range of: about 0.01 mg/ml to about 1.5 mg/ml, about 0.01 mg/ml to about 1.3 mg/ml, about 0.01 mg/ml to about 1 mg/ml, about 0.01 mg/ml to about 0.8 mg/ml, about 0.01 mg/ml to about 0.5 mg/ml, about 0.01 mg/ml to about 0.3 mg/ml, about 0.01 mg/ml to about 0.2 mg/ml, about 0.03 mg/ml to about 1.5 mg/ml, about 0.03 mg/ml to about 1.3 mg/ml, about 0.03 mg/ml to about 1 mg/ml, about 0.03 mg/ml to about 0.8 mg/ml, about 0.03 mg/ml to about 0.5 mg/ml, about 0.03 mg/ml to about 0.3 mg/ml, about 0.03 mg/ml to about 0.2 mg/ml, about 0.05 mg/ml to about 1.5 mg/ml, about 0.05 mg/ml to about 1.3 mg/ml, about 0.05 mg/ml to about 1 mg/ml, about 0.05 mg/ml to about 0.8 mg/ml, about 0.05 mg/ml to about 0.5 mg/ml, about 0.05 mg/ml to about 0.3 mg/ml, about 0.05 mg/ml to about 0.2 mg/ml, about 0.08 mg/ml to about 1.5 mg/ml, about 0.08 mg/ml to about 1.3 mg/ml, about 0.08 mg/ml to about 1 mg/ml, about 0.08 mg/ml to about 0.8 mg/ml, about 0.08 mg/ml to about 0.5 mg/ml, about 0.08 mg/ml to about 0.3 mg/ml, about 0.08 mg/ml to about 0.2 mg/ml, about 0.1 mg/ml to about 1.5 mg/ml, about 0.1 mg/ml to about 1.3 mg/ml, about 0.1 mg/ml to about 1 mg/ml, about 0.1 mg/ml to about 0.8 mg/ml, about 0.1 mg/ml to about 0.5 mg/ml, about 0.1 mg/ml to about 0.3 mg/ml, or about 0.1 mg/ml to about 0.2 mg/ml.

In some embodiments, the preservation composition may be free of, or comprise, vitamin B12 in the following concentration range: about 0.01 mg/ml to about 1.5 mg/ml, about 0.01 mg/ml to about 1.3 mg/ml, about 0.01 mg/ml to about 1 mg/ml, about 0.01 mg/ml to about 0.8 mg/ml, about 0.01 mg/ml to about 0.5 mg/ml, about 0.01 mg/ml to about 0.3 mg/ml, about 0.01 mg/ml to about 0.2 mg/ml, about 0.03 mg/ml to about 1.5 mg/ml, about 0.03 mg/ml to about 1.3 mg/ml, about 0.03 mg/ml to about 1 mg/ml, about 0.03 mg/ml to about 0.8 mg/ml, about 0.03 mg/ml to about 0.5 mg/ml, about 0.03 mg/ml to about 0.3 mg/ml, about 0.03 mg/ml to about 0.2 mg/ml, about 0.05 mg/ml to about 1.5 mg/ml, about 0.05 mg/ml to about 1.3 mg/ml, about 0.05 mg/ml to about 1 mg/ml, about 0.05 mg/ml to about 0.8 mg/ml, about 0.05 mg/ml to about 0.5 mg/ml, about 0.05 mg/ml to about 0.3 mg/ml, about 0.05 mg/ml to about 0.2 mg/ml, about 0.08 mg/ml to about 1.5 mg/ml, about 0.08 mg/ml to about 1.3 mg/ml, about 0.08 mg/ml to about 1 mg/ml, about 0.08 mg/ml to about 0.8 mg/ml, about 0.08 mg/ml to about 0.5 mg/ml, about 0.08 mg/ml to about 0.3 mg/ml, about 0.08 mg/ml to about 0.2 mg/ml, about 0.1 mg/ml to about 1.5 mg/ml, about 0.1 mg/ml to about 1.3 mg/ml, about 0.1 mg/ml to about 1 mg/ml, about 0.1 mg/ml to about 0.8 mg/ml, about 0.1 mg/ml to about 0.5 mg/ml, about 0.1 mg/ml to about 0.3 mg/ml, or about 0.1 mg/ml to about 0.2 mg/ml.

In some embodiments, the preservation composition may be free of, or comprise, alanine in the following concentration range: about 1 mg/ml to about 200 mg/ml, about 1 mg/ml to about 180 mg/ml, about 1 mg/ml to about 150 mg/ml, about 1 mg/ml to about 120 mg/ml, about 1 mg/ml to about 100 mg/ml, about 1 mg/ml to about 80 mg/ml, about 1 mg/ml to about 50 mg/ml, about 2 mg/ml to about 200 mg/ml, about 2 mg/ml to about 180 mg/ml, about 2 mg/ml to about 150 mg/ml, about 2 mg/ml to about 120 mg/ml, about 2 mg/ml to about 100 mg/ml, about 2 mg/ml to about 80 mg/ml, about 2 mg/ml to about 50 mg/ml, about 3 mg/ml to about 200 mg/ml, about 3 mg/ml to about 180 mg/ml, about 3 mg/ml to about 150 mg/ml, about 3 mg/ml to about 120 mg/ml, about 3 mg/ml to about 100 mg/ml, about 3 mg/ml to about 80 mg/ml, about 3 mg/ml to about 50 mg/ml, about 4 mg/ml to about 200 mg/ml, about 4 mg/ml to about 180 mg/ml, about 4 mg/ml to about 150 mg/ml, about 4 mg/ml to about 120 mg/ml, about 4 mg/ml to about 100 mg/ml, about 4 mg/ml to about 80 mg/ml, or about 4 mg/ml to about 50 mg/ml.

In some embodiments, the preservation composition may be free of, or comprise, arginine in the following concentration range: about 1 mg/ml to about 200 mg/ml, about 1 mg/ml to about 180 mg/ml, about 1 mg/ml to about 150 mg/ml, about 1 mg/ml to about 120 mg/ml, about 1 mg/ml to about 100 mg/ml, about 1 mg/ml to about 80 mg/ml, about 1 mg/ml to about 50 mg/ml, about 2 mg/ml to about 200 mg/ml, about 2 mg/ml to about 180 mg/ml, about 2 mg/ml to about 150 mg/ml, about 2 mg/ml to about 120 mg/ml, about 2 mg/ml to about 100 mg/ml, about 2 mg/ml to about 80 mg/ml, about 2 mg/ml to about 50 mg/ml, about 3 mg/ml to about 200 mg/ml, about 3 mg/ml to about 180 mg/ml, about 3 mg/ml to about 150 mg/ml, about 3 mg/ml to about 120 mg/ml, about 3 mg/ml to about 100 mg/ml, about 3 mg/ml to about 80 mg/ml, about 3 mg/ml to about 50 mg/ml, about 4 mg/ml to about 200 mg/ml, about 4 mg/ml to about 180 mg/ml, about 4 mg/ml to about 150 mg/ml, about 4 mg/ml to about 120 mg/ml, about 4 mg/ml to about 100 mg/ml, about 4 mg/ml to about 80 mg/ml, or about 4 mg/ml to about 50 mg/ml.

In some embodiments, the preservation composition may be free of, or comprise, aspartic acid in the following concentration range: about 0.2 mg/ml to about 80 mg/ml, about 0.2 mg/ml to about 50 mg/ml, about 0.2 mg/ml to about 30 mg/ml, about 0.2 mg/ml to about 20 mg/ml, about 0.5 mg/ml to about 80 mg/ml, about 0.5 mg/ml to about 50 mg/ml, about 0.5 mg/ml to about 30 mg/ml, about 0.5 mg/ml to about 20 mg/ml, about 0.9 mg/ml to about 80 mg/ml, about 0.9 mg/ml to about 50 mg/ml, about 0.9 mg/ml to about 30 mg/ml, about 0.9 mg/ml to about 20 mg/ml, about 1 mg/ml to about 80 mg/ml, about 1 mg/ml to about 50 mg/ml, about 1 mg/ml to about 30 mg/ml, or about 1 mg/ml to about 20 mg/ml.

In some embodiments, the preservation composition may be free of, or comprise, cystine in the following concentration range: about 0.01 mg/ml to about 3 mg/ml, about 0.01 mg/ml to about 2.8 mg/ml, about 0.01 mg/ml to about 2.5 mg/ml, about 0.01 mg/ml to about 2.2 mg/ml, about 0.01 mg/ml to about 2 mg/ml, about 0.01 mg/ml to about 1.8 mg/ml, about 0.01 mg/ml to about 1.5 mg/ml, about 0.01 mg/ml to about 1.3 mg/ml, about 0.01 mg/ml to about 1 mg/ml, about 0.03 mg/ml to about 3 mg/ml, about 0.03 mg/ml to about 2.8 mg/ml, about 0.03 mg/ml to about 2.5 mg/ml, about 0.03 mg/ml to about 2.2 mg/ml, about 0.03 mg/ml to about 2 mg/ml, about 0.03 mg/ml to about 1.8 mg/ml, about 0.03 mg/ml to about 1.5 mg/ml, about 0.03 mg/ml to about 1.3 mg/ml, about 0.03 mg/ml to about 1 mg/ml, about 0.04 mg/ml to about 3 mg/ml, about 0.04 mg/ml to about 2.8 mg/ml, about 0.04 mg/ml to about 2.5 mg/ml, about 0.04 mg/ml to about 2.2 mg/ml, about 0.04 mg/ml to about 2 mg/ml, about 0.04 mg/ml to about 1.8 mg/ml, about 0.04 mg/ml to about 1.5 mg/ml, about 0.04 mg/ml to about 1.3 mg/ml, about 0.04 mg/ml to about 1 mg/ml, about 0.1 mg/ml to about 3 mg/ml, about 0.03 mg/ml to about 3 mg/ml, about 0.03 mg/ml to about 2.8 mg/ml, about 0.03 mg/ml to about 2.5 mg/ml, about 0.03 mg/ml to about 2.2 mg/ml, about 0.03 mg/ml to about 2 mg/ml, about 0.03 mg/ml to about 1.8 mg/ml, about 0.03 mg/ml to about 1.5 mg/ml, about 0.03 mg/ml to about 1.3 mg/ml, about 0.03 mg/ml to about 1 mg/ml, about 0.04 mg/ml to about 3 mg/ml, about 0.04 mg/ml to about 2.8 mg/ml, about 0.04 mg/ml to about 2.5 mg/ml, about 0.04 mg/ml to about 2.2 mg/ml, about 0.04 mg/ml to about 2 mg/ml, about 0.04 mg/ml to about 1.8 mg/ml, about 0.04 mg/ml to about 1.5 mg/ml, about 0.04 mg/ml to about 1.3 mg/ml, about 0.04 mg/ml to about 1 mg/ml, about 0.1 mg/ml to about 2.8 mg/ml, about 0.1 mg/ml to about 2.5 mg/ml, about 0.1 mg/ml to about 2.2 mg/ml, about 0.1 mg/ml to about 2 mg/ml, about 0.1 mg/ml to about 1.8 mg/ml, about 0.1 mg/ml to about 1.5 mg/ml, about 0.1 mg/ml to about 1.3 mg/ml, about 0.1 mg/ml to about 1 mg/ml, about 0.3 mg/ml to about 3 mg/ml, about 0.3 mg/ml to about 2.8 mg/ml, about 0.3 mg/ml to about 2.5 mg/ml, about 0.3 mg/ml to about 2.2 mg/ml, about 0.3 mg/ml to about 2 mg/ml, about 0.3 mg/ml to about 1.8 mg/ml, about 0.3 mg/ml to about 1.5 mg/ml, about 0.3 mg/ml to about 1.3 mg/ml, about 0.3 mg/ml to about 1 mg/ml, about 0.5 mg/ml to about 3 mg/ml, about 0.5 mg/ml to about 2.8 mg/ml, about 0.5 mg/ml to about 2.5 mg/ml, about 0.5 mg/ml to about 2.2 mg/ml, about 0.5 mg/ml to about 2 mg/ml, about 0.5 mg/ml to about 1.8 mg/ml, about 0.5 mg/ml to about 1.5 mg/ml, about 0.5 mg/ml to about 1.3 mg/ml, about 0.5 mg/ml to about 1 mg/ml, about 0.8 mg/ml to about 3 mg/ml, about 0.8 mg/ml to about 2.8 mg/ml, about 0.8 mg/ml to about 2.5 mg/ml, about 0.8 mg/ml to about 2.2 mg/ml, about 0.8 mg/ml to about 2 mg/ml, about 0.8 mg/ml to about 1.8 mg/ml, about 0.8 mg/ml to about 1.5 mg/ml, about 0.8 mg/ml to about 1.3 mg/ml, or about 0.8 mg/ml to about 1 mg/ml.

In some embodiments, the preservation composition may be free of, or comprise, glutamic acid in the following concentration range: about 0.5 mg/ml to about 100 mg/ml, about 0.5 mg/ml to about 80 mg/ml, about 0.5 mg/ml to about 50 mg/ml, about 0.5 mg/ml to about 40 mg/ml, about 0.8 mg/ml to about 100 mg/ml, about 0.8 mg/ml to about 80 mg/ml, about 0.8 mg/ml to about 50 mg/ml, about 0.8 mg/ml to about 40 mg/ml, about 1 mg/ml to about 100 mg/ml, about 1 mg/ml to about 80 mg/ml, about 1 mg/ml to about 50 mg/ml, about 1 mg/ml to about 40 mg/ml, about 1.5 mg/ml to about 100 mg/ml, about 1.5 mg/ml to about 80 mg/ml, about 1.5 mg/ml to about 50 mg/ml, or about 1.5 mg/ml to about 40 mg/ml.

In some embodiments, the preservation composition may be free of, or comprise, glycine in the following concentration range: about 0.5 mg/ml to about 100 mg/ml, about 0.5 mg/ml to about 80 mg/ml, about 0.5 mg/ml to about 50 mg/ml, about 0.5 mg/ml to about 40 mg/ml, about 0.8 mg/ml to about 100 mg/ml, about 0.8 mg/ml to about 80 mg/ml, about 0.8 mg/ml to about 50 mg/ml, about 0.8 mg/ml to about 40 mg/ml, about 1 mg/ml to about 100 mg/ml, about 1 mg/ml to about 80 mg/ml, about 1 mg/ml to about 50 mg/ml, about 1 mg/ml to about 40 mg/ml, about 1.5 mg/ml to about 100 mg/ml, about 1.5 mg/ml to about 80 mg/ml, about 1.5 mg/ml to about 50 mg/ml, about 1.5 mg/ml to about 40 mg/ml, about 2 mg/ml to about 100 mg/ml, about 2 mg/ml to about 80 mg/ml, about 2 mg/ml to about 50 mg/ml, or about 2 mg/ml to about 40 mg/ml.

In some embodiments, the preservation composition may be free of, or comprise, histidine in the following concentration range: about 0.5 mg/ml to about 100 mg/ml, about 0.5 mg/ml to about 80 mg/ml, about 0.5 mg/ml to about 50 mg/ml, about 0.5 mg/ml to about 40 mg/ml, about 0.8 mg/ml to about 100 mg/ml, about 0.8 mg/ml to about 80 mg/ml, about 0.8 mg/ml to about 50 mg/ml, about 0.8 mg/ml to about 40 mg/ml, about 1 mg/ml to about 100 mg/ml, about 1 mg/ml to about 80 mg/ml, about 1 mg/ml to about 50 mg/ml, about 1 mg/ml to about 40 mg/ml, about 1.5 mg/ml to about 100 mg/ml, about 1.5 mg/ml to about 80 mg/ml, about 1.5 mg/ml to about 50 mg/ml, or about 1.5 mg/ml to about 40 mg/ml.

In some embodiments, the preservation composition may be free of, or comprise, isoleucine in the following concentration range: about 0.5 mg/ml to about 100 mg/ml, about 0.5 mg/ml to about 80 mg/ml, about 0.5 mg/ml to about 50 mg/ml, about 0.5 mg/ml to about 40 mg/ml, about 0.8 mg/ml to about 100 mg/ml, about 0.8 mg/ml to about 80 mg/ml, about 0.8 mg/ml to about 50 mg/ml, about 0.8 mg/ml to about 40 mg/ml, about 1 mg/ml to about 100 mg/ml, about 1 mg/ml to about 80 mg/ml, about 1 mg/ml to about 50 mg/ml, about 1 mg/ml to about 40 mg/ml, about 1.5 mg/ml to about 100 mg/ml, about 1.5 mg/ml to about 80 mg/ml, about 1.5 mg/ml to about 50 mg/ml, or about 1.5 mg/ml to about 40 mg/ml.

In some embodiments, the preservation composition may be free of, or comprise, methionine in the following concentration range: about 0.5 mg/ml to about 100 mg/ml, about 0.5 mg/ml to about 80 mg/ml, about 0.5 mg/ml to about 50 mg/ml, about 0.5 mg/ml to about 40 mg/ml, about 0.8 mg/ml to about 100 mg/ml, about 0.8 mg/ml to about 80 mg/ml, about 0.8 mg/ml to about 50 mg/ml, about 0.8 mg/ml to about 40 mg/ml, about 1 mg/ml to about 100 mg/ml, about 1 mg/ml to about 80 mg/ml, about 1 mg/ml to about 50 mg/ml, about 1 mg/ml to about 40 mg/ml, about 1.5 mg/ml to about 100 mg/ml, about 1.5 mg/ml to about 80 mg/ml, about 1.5 mg/ml to about 50 mg/ml, or about 1.5 mg/ml to about 40 mg/ml.

In some embodiments, the preservation composition may be free of, or comprise, leucine in the following concentration range: about 1 mg/ml to about 200 mg/ml, about 1 mg/ml to about 180 mg/ml, about 1 mg/ml to about 150 mg/ml, about 1 mg/ml to about 120 mg/ml, about 1 mg/ml to about 100 mg/ml, about 1 mg/ml to about 80 mg/ml, about 1 mg/ml to about 50 mg/ml, about 2 mg/ml to about 200 mg/ml, about 2 mg/ml to about 180 mg/ml, about 2 mg/ml to about 150 mg/ml, about 2 mg/ml to about 120 mg/ml, about 2 mg/ml to about 100 mg/ml, about 2 mg/ml to about 80 mg/ml, about 2 mg/ml to about 50 mg/ml, about 3 mg/ml to about 200 mg/ml, about 3 mg/ml to about 180 mg/ml, about 3 mg/ml to about 150 mg/ml, about 3 mg/ml to about 120 mg/ml, about 3 mg/ml to about 100 mg/ml, about 3 mg/ml to about 80 mg/ml, about 3 mg/ml to about 50 mg/ml, about 4 mg/ml to about 200 mg/ml, about 4 mg/ml to about 180 mg/ml, about 4 mg/ml to about 150 mg/ml, about 4 mg/ml to about 120 mg/ml, about 4 mg/ml to about 100 mg/ml, about 4 mg/ml to about 80 mg/ml, or about 4 mg/ml to about 50 mg/ml.

In some embodiments, the preservation composition may be free of, or comprise, phenylalanine in the following concentration range: about 1 mg/ml to about 200 mg/ml, about 1 mg/ml to about 180 mg/ml, about 1 mg/ml to about 150 mg/ml, about 1 mg/ml to about 120 mg/ml, about 1 mg/ml to about 100 mg/ml, about 1 mg/ml to about 80 mg/ml, about 1 mg/ml to about 50 mg/ml, about 2 mg/ml to about 200 mg/ml, about 2 mg/ml to about 180 mg/ml, about 2 mg/ml to about 150 mg/ml, about 2 mg/ml to about 120 mg/ml, about 2 mg/ml to about 100 mg/ml, about 2 mg/ml to about 80 mg/ml, about 2 mg/ml to about 50 mg/ml, about 3 mg/ml to about 200 mg/ml, about 3 mg/ml to about 180 mg/ml, about 3 mg/ml to about 150 mg/ml, about 3 mg/ml to about 120 mg/ml, about 3 mg/ml to about 100 mg/ml, about 3 mg/ml to about 80 mg/ml, about 3 mg/ml to about 50 mg/ml, about 4 mg/ml to about 200 mg/ml, about 4 mg/ml to about 180 mg/ml, about 4 mg/ml to about 150 mg/ml, about 4 mg/ml to about 120 mg/ml, about 4 mg/ml to about 100 mg/ml, about 4 mg/ml to about 80 mg/ml, or about 4 mg/ml to about 50 mg/ml.

In some embodiments, the cells are present in the preservation composition in the following concentration range: about 10⁴ cells/ml to about 10⁸ cells/ml, about 10⁵ cells/ml to about 10⁷ cells/ml, about 10⁵ cells/ml to about 10⁸ cells/ml, about 10⁴ cells/ml to about 10⁷ cells/ml, about 10⁵ cells/ml, about 10⁶ cells/ml, or about 10⁷ cells/ml. The concentration of the cells in the preservation composition may be higher than 10⁸ cells/ml or lower than 10⁴ cells/ml. In some embodiments, the concentration of the cells in the preservation composition may vary depending on the cell type. A skilled person can determine the concentration of a particular type of cells.

In some embodiments, the cells have at least or about 50%, at least or about 55%, at least or about 60%, at least or about 65%, at least or about 70%, at least or about 75%, at least or about 80%, at least or about 85%, at least or about 90%, or at least or about 95% viability after storage or preservation.

The present disclosure provides a method for preserving a biological material. The method may comprise the following steps: (a) combining, mixing, or contacting the preservation composition of the present invention with the biological material; (b) cooling the mixture; and (c) storing the biological material (e.g., under appropriate storage conditions).

In some embodiments, the composition of the present invention has a pH value of about 6.0 to about 8.5, about 6.5 to about 8, about 6.9 to about 7.5, about 6.5 to about 7.5, or about 7.2 to about 7.4 at room temperature or ambient temperature (e.g., at 25° C.).

The term “preservation” refers to a process whereby a biological material is maintained under conditions in which the biological material exhibits substantially decreased biological activity but still retains its viability, and, when released from the preservation state, can resume to a substantially normal biological activity.

The term “preservation composition” relates to a composition (or a composition when diluted or dissolved) that allows for the preservation of a biological material such that the biological material retains its viability. One specific embodiment of the preservation composition is a composition for preserving a biological material at a low temperature. In some embodiments, the preservation composition is a preservation solution.

The biological material (e.g., the cells) may be preserved at a temperature below the physiological temperature but above the freezing point (e.g., about 0° C. to about 10° C., about 0° C. to about 8° C., about 0° C. to about 4° C., about 2° C. to about 8° C., or about 4° C. to about 8° C.), at which temperature the biological process slows down, thereby allowing for long-term storage of the biological material.

The biological material (e.g., the cells) can be preserved for at least or about 3 hours, at least or about 6 hours, at least or about 12 hours, at least or about 24 hours, at least or about 48 hours, at least or about 72 hours, at least or about 96 hours, at least or about 1 day, at least or about 2 days, at least or about 3 days, at least or about 4 days, at least or about 5 days, at least or about 6 days, at least or about 7 days, at least or about 1 week, at least or about 2 weeks, at least or about 3 weeks, at least or about 4 weeks or longer, or any time period.

The term “about” with respect to a numerical value refers to #10% of the numerical value. In other words, the numerical value may be within the range from 90% of the value to 110% of the value.

The preservation composition of the present invention may be a liquid or solid. In some embodiments, the preservation composition of present invention is a concentrated composition, for example, in a dry form (e.g., powder, tablet, granule, or any other appropriate physical form) or in a liquid form, e.g., a 2-fold, 3-fold, 4-fold, 5-fold, 6-fold, 7-fold, 8-fold, 9-fold, 10-fold, 15-fold, or 20-fold stock solution. The stock solution may be diluted by a factor of 2, 3, 4, 5, 6, 7, 8, 9, 10, 15, 20, etc., using, e.g., a culture medium, a physiological solution, a buffer, water, etc. The preservation composition in a dry form may be converted to a liquid form by adding, e.g., a culture medium, a physiological solution, a buffer, water, etc. (e.g., by dissolving in e.g., the culture medium, the physiological solution, the buffer, water, etc.)

As used herein, the term “viability” refers to the percentage of a viable biological material (e.g., cells, for example, based on the presence of DNA and/or an intact cell membrane system). In some embodiments, the viable biological material refers to a biological material comprising some live cells or cell portions which, upon being released from the preservation state, have a metabolic activity, or will become metabolically active.

In some embodiments, the composition and method of the present invention ensure that the cells display a limited amount of, or minimal, necrosis and apoptosis after storage or preservation. In some embodiments, the necrosis and/or cell apoptosis is observed to be less than or about 25% of the cells, less than or about 20% of the cells, less than or about 15% of the cells, less than or about 10% of the cells, less than or about 5% of the cells, or less than or about 1% of the cells.

The viability may be measured by any method known in the art. In some embodiments, the viability is measured using a Trypan blue internalization test or by measuring propidium iodide uptake. In some embodiments, the viability is measured by assaying the ability of the cells to attach effectively (e.g., an attachment assay). In some embodiments, a proliferation assay may be used to determine whether the attached cells can proliferate as expected after preservation. The attachment and proliferation efficiencies may be compared with those of control cells not having been preserved.

There are various tests known in the art to determine the viability and function of the cells. In some embodiments, these tests are dependent on the cell type and the desired use of the cells.

For stem cells or progenitor cells, the methods described herein can further ensure that the cells retain their pluripotency. This may be determined by determining the expression of a lineage-specific marker. For example, functional characterization of mesenchymal stem cells may include in vitro induction of adipogenic, osteogenic and chondrogenic differentiation using commercially available differentiation kits and RT-PCR to detect lineage-specific expression of mRNA, so as to indicate the adipogenic, osteogenic, and chondrogenic differentiation potential. Similarly, the quality of undifferentiated stem cells may be tested by isolating mRNA and testing on cell-specific markers. In particular embodiments, the ability to differentiate into a cell of the specified lineage is maintained, i.e., does not significantly differ from unprocessed cells. Several in vitro methods may be used to assess the recovery of stem cells after experimental treatment. These assessments may include, but are not limited to, membrane integrity, metabolic and other functional assays, and/or colony growth in culture, and fluorescence assays, such as SYTO/EB. In some embodiments, differentiation tests, immunophenotypic characterization, and/or an inspection of the morphology may be used to assay stem cells and/or progenitor cells.

In some embodiments, the preservation composition and method of the present invention, as well as the biological material recovered from preservation using the preservation composition and method of the present invention, may be used in research and/or clinical applications (e.g., cell-based therapies, transplantation, regenerative medicine, diagnostics and genetic testing, cell/tissue banking for surveillance, toxicity testing, and for in vitro fertilization).

The present invention will be further described below in conjunction with specific examples. It should be understood that these examples are used only to describe the present invention without limiting the scope of the present invention. Experimental methods for which specific conditions are not specified in the following examples are generally carried out in accordance with conventional conditions, such as the conditions described in Sambrook et al., Molecular Cloning: A Laboratory Manual (New York: Cold Spring Harbor Laboratory Press (1989)), or in accordance with conditions suggested by the manufacturers.

### EXAMPLES

### Preparation of the Preservation Solution

The preservation solution was prepared using the components and amounts indicated in Table 1, and was labeled as CE01KS.

TABLE 1
Formula of the CEO1KS cell preservation solution (formulated
according to a total volume of 250 ml)

| | | | Reagent formulation |
|---|---|---|---|
| | No. | Reagent used | regimen volume |
| CE01KS | 1 | Compound | 215-240 ml |
| cell | | electrolyte | |
| preservation | | injection | |
| solution | 2 | Glucose-sodium | 8-30 ml |
| | | chloride injection | |
| | 3 | Amino acid | 0.59 ml-5.9 ml |
| | 4 | Water-soluble | Powdered; after each tube |
| | | vitamin | of the vitamin was dissolved |
| | | | with 5 ml of the glucose- |
| | | | sodium chloride injection, |
| | | | 1/47 to 1/24 tube was |
| | | | taken according to the |
| | | | volume ratio |

Every 1000 ml of the compound electrolyte injection comprises: 5.26 g of sodium chloride, 5.02 g of sodium gluconate, 3.68 g of sodium acetate, 0.37 g of potassium chloride, and 0.30 g of magnesium chloride.

Every 100 ml of the glucose-sodium chloride injection comprises 5 g of glucose and 0.9 g of sodium chloride.

250 ml of the amino acid comprises: 3.05 g of alanine, 2.10 g of arginine, 0.63 g of aspartic acid, 0.05 g of cystine, 1.05 g of glutamic acid, 1.48 g of glycine, 1.2 g of histidine, 1.48 g of leucine, 1.05 g of isoleucine, 1.05 g of methionine, and 1.48 g of phenylalanine.

Each tube of the water-soluble vitamin comprises: 3.1 mg of thiamine nitrate, 4.9 mg of riboflavin sodium phosphate, 40 mg of niacinamide, 4.9 mg of pyridoxine hydrochloride, 16.5 mg of sodium pantothenate, 113 mg of vitamin C sodium, 60 μg of biotin, 0.4 mg of folate, and 5.0 μg of vitamin B12.

### Preparation of Cell Suspensions in CE01KS and Physiological Saline

1) Cell resuscitation: 30 ml of CE01KS and 30 ml of physiological saline were measured, and were pre-heated in a water bath at 37° C. for 10 min in advance. Two tubes of cryopreserved adipose-derived mesenchymal stem cells were taken, and the cells were resuscitated using the CE01KS and physiological saline respectively. 300 ul were taken for counting.

2) Cell centrifugation: The resuscitated cells were placed in a pre-cooled centrifuge at 4° C. and centrifuged at 800-1000 rpm for 5-10 min;

3) Cell washing: The supernatant was discarded. The cells were resuspended, and were added with 30 ml of either of the above two kinds of liquid for washing. Centrifugation was performed according to the cell centrifugation manner described above. The supernatant above the cells were again discarded. 300 ul were taken for counting.

4) Filtering: The bottom-layer precipitate was resuspended, and was added with either of the above two kinds of liquid to 30 ml. Large masses of cells were removed using a 100 um filter. 300 ul were taken for counting.

5) Counting using an NC200 cell counter: Operation was performed exactly in accordance with the operating manual of the cell counter. 50 ul of cell suspension were mixed 1:1 with kit solution 1. The mixture was sampled and loaded in a Vial, and “Viability and Cell Count-Aggregated Cells Assay” was selected. The primary liquid without the solution added was sampled and loaded in another new Vial. Cell number was read.

6) Bringing cell suspension to volume: After cell filtration, centrifugation was performed according to step 2, and the cells were resuspended at 1.3*10⁷/ml.

7) Live cell, necrosis and apoptosis proportions, and cell viability, cell surface markers, and cell morphology

Cell apoptosis detection: The cells were dispensed and vialed, and the time point was set as 0 h. The cells were placed in a refrigerator at 2° C. to 8° C., and 60 ul of the cells were taken at 0 h, 24 h, and 48 h respectively. The cells were washed with 1% HSA DPBS twice. Then, 5 ul of FITC Annexin V were added to the cells in each flow tube, and the flow tube was placed in dark at room temperature for 15 min. 5 ul of Propidium Iodide were added, and detection was performed using a Miltenyi MACSQuant Analyzer 10.

Detection of cell surface markers: 60 ul of the cells were taken at 0 h, 24 h, and 48 h respectively. The cells were washed with 1% HSA-DPBS washing solution once, and then dispensed into 3 tubes. After the cells were further washed once, the first tube was added with 1 ul of each of HLA-DR-PE, CD14-FITC, and CD45-APC, the second tube was added with 1 ul of each of CD73-PE, CD90-FITC, and CD105-APC, and the third tube was added with 1 ul of each of PE, FITC, and APC ios. The cells were incubated at 4° C. for 40 min. After being washed, the cells were added with 7AAD and incubated for 10 min, and were subjected to detection on the Miltenyi MACSQuant Analyzer 10.

6*10⁵ cells were taken and added to 6 ml of CBMG culture medium at 0 h, 24 h, and 48 h respectively. The cells were inoculated into three wells, 2 ml each well. After inoculation, the cells were further cultured, and photos were taken every day.

### Results

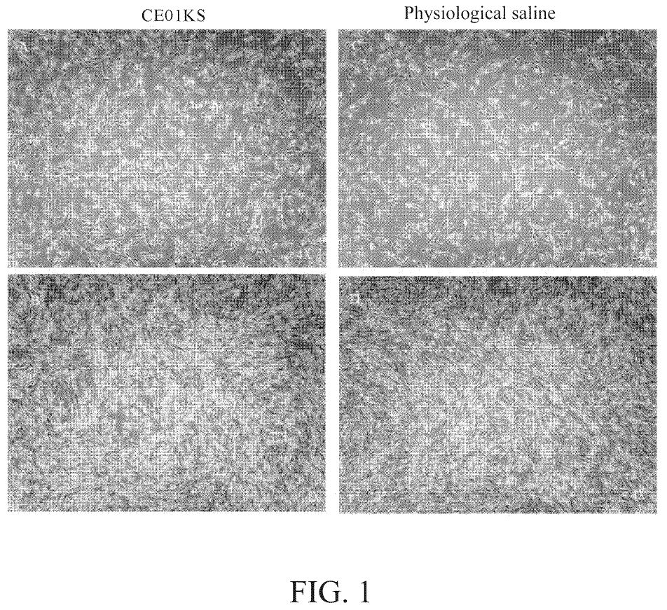

The results of the viability of the cells having been preserved are as shown in Table 2, and the cell morphologies are as shown in FIGS. 1-3.

TABLE 2
Change in the viability of the cells having been preserved

| | CEO1KS (%) | Physiological saline (%) |
|---|---|---|
| 0 h | 92.10 | 94.00 |
| 24 h | 84.30 | 79.30 |
| 48 h | 72.00 | 53.90 |
| 72 h | 73.80 | 32.10 |

The change in cell viability can be seen from the Table: At 0 h, the cell viability was above 90%. After being placed for 72 h, the viability of cells having been preserved with CE01KS remained above 70%, while the cells having been preserved with physiological saline was lower than 35%.

FIG. 1 shows images of cell morphology of cells having been preserved for 0 h, wherein A: cell morphology of cells having been preserved in CE01KS for 0 h, on day 1 after inoculation; B: cell morphology of cells having been preserved in CE01KS for 0 h, on day 3 after inoculation; C:
cell morphology of cells having been preserved in physiological saline for 0 h, on day 1 after inoculation; and D: cell morphology of cells having been preserved in physiological saline for 0 h, on day 3 after inoculation.

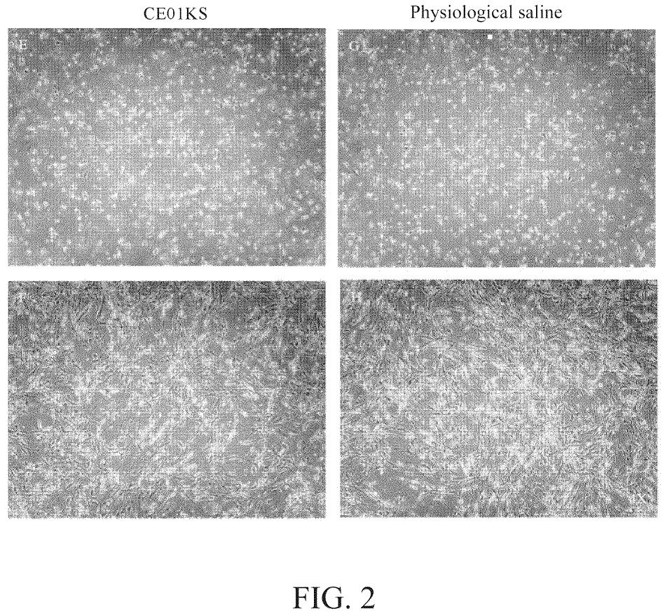

FIG. 2 shows images of cell morphology of cells having been preserved for 24 h, wherein E: cell morphology of cells having been preserved in CE01KS in a refrigerator for 24 h, on day 1 after inoculation; F: cell morphology of cells having been preserved in CE01KS in a refrigerator for 24 h, on day 4 after inoculation; G: cell morphology of cells having been preserved in physiological saline in a refrigerator for 24 h, on day 1 after inoculation; and H: cell morphology of cells having been preserved in physiological saline in a refrigerator for 24 h, on day 4 after inoculation.

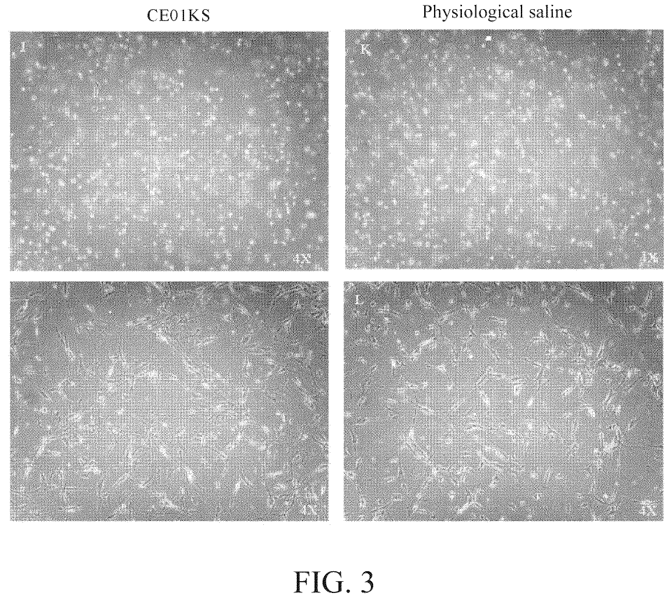

FIG. 3 shows images of cell morphology of cells having been preserved for 48 h, wherein I: cell morphology of cells having been preserved in CE01KS in a refrigerator for 48 h, on day 1 after inoculation; K: cell morphology of cells having been preserved in CE01KS in a refrigerator for 48 h, on day 6 after inoculation; J: cell morphology of cells having been preserved in physiological saline in a refrigerator for 48 b, on day 1 after inoculation; and L: cell morphology of cells having been preserved in physiological saline in a refrigerator for 48 h, on day 6 after inoculation.

TABLE 3
Apoptosis of cells after being placed in a refrigerator for 0 h, 24 h, 48 h, and 72 h

| | | CE01KS | | | Physiological saline | | |
|---|---|---|---|---|---|---|---|
| Time point | | Live cells | Apoptosis | Necrosis | Live cells | Apoptosis | Necrosis |
| 0 | h | 89.21% | 1.45% | 8.49% | 86.84% | 2.99% | 9.74% |
| 24 | h | 72.84% | 0.53% | 23.74% | 71.81% | 0.65% | 26.08% |
| 48 | h | 72.17% | 0.43% | 23.26% | 46.64% | 0.45% | 46.59% |
| 72 | h | 64.57% | 0.09% | 19.76% | 34.21% | 0.01% | 22.43% |

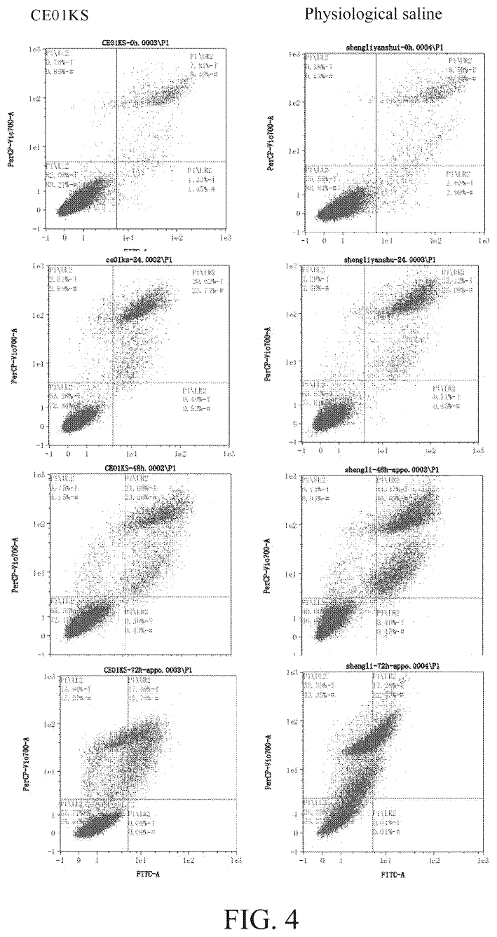

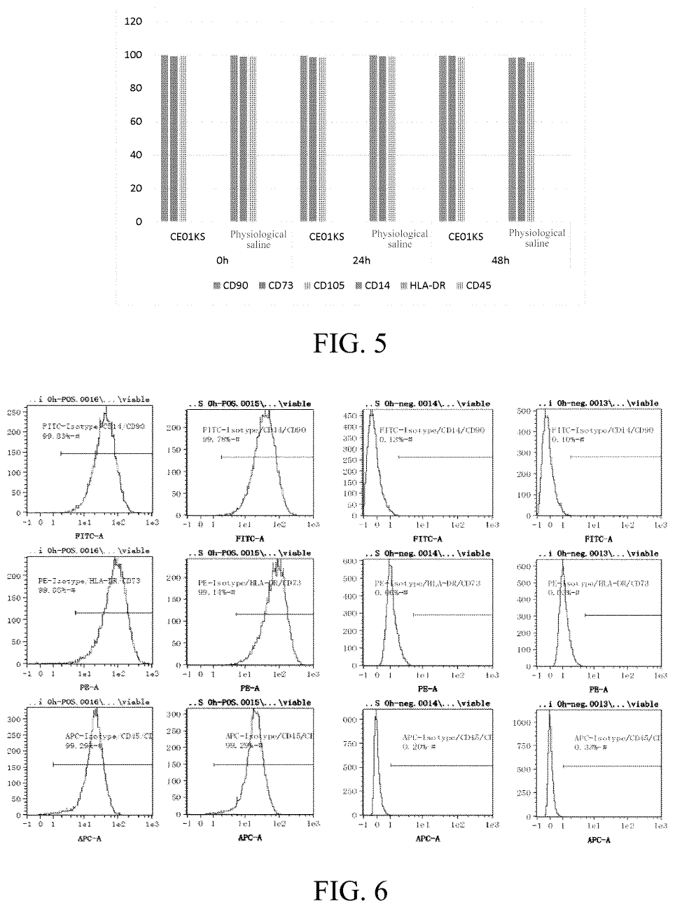

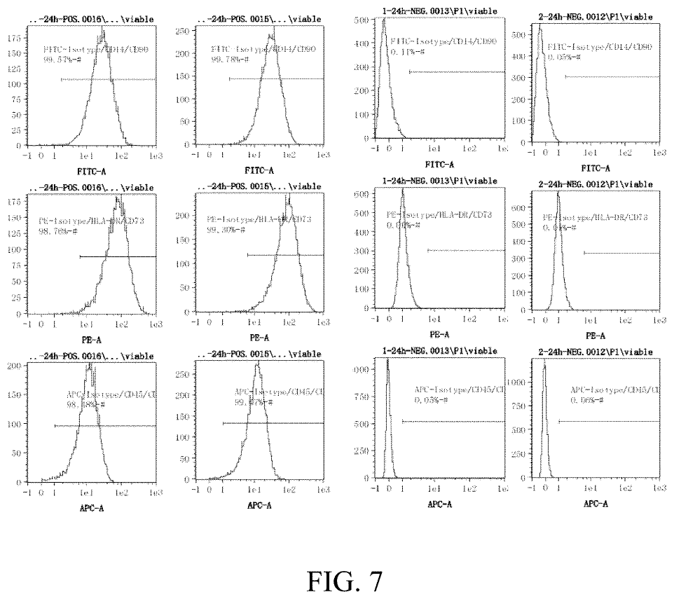

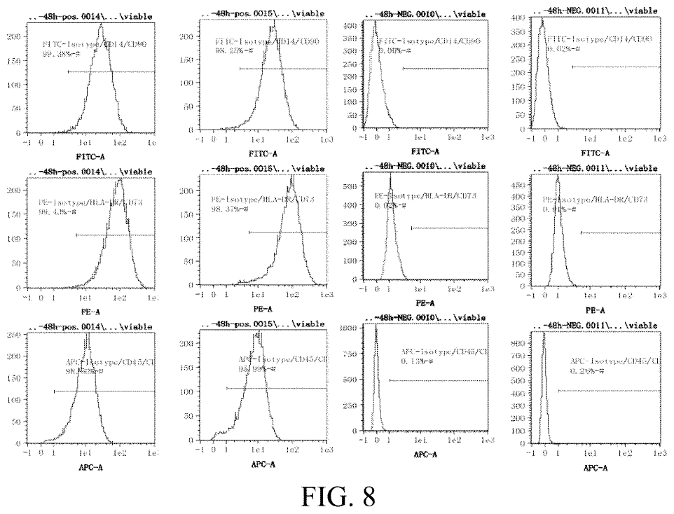

The results of cell apoptosis are as shown in Table 3, in which the apoptosis of the cells upon dispensing at 0 h, and after being placed for 24 h, 48 h, and 72 h can be seen. From dispensing at 0 h to 72 h of placement, the proportion of live cells decreased and the proportion of necrotic cells accordingly increased over time. However, the cells having been preserved with CE01KS showed a relatively slow decrease in the proportion of live cells, and could maintain cell viability well; while the cells have been preserved with physiological saline showed a relatively fast decrease in the proportion of live cells, and could only maintain a cell viability of more than 70% for 24 h. The details of cell apoptosis are as shown in FIG. 4.

TABLE 4
Change in surface markers of the cells after being
placed in a refrigerator for 0 h, 24 h, and 48 h

| Time | | | | | | | | |
|---|---|---|---|---|---|---|---|---|
| point | | Group | CD90 | CD73 | CD105 | CD14 | HLA-DR | CD45 |
| 0 | h | CE01KS | 99.72% | 99.08% | 99.16% | 0.07% | 0.00% | 0.07% |
| | | Physiological | 99.61% | 98.90% | 99.10% | 0.00% | 0.00% | 0.14% |
| | | saline | | | | | | |
| 24 | h | CE01KS | 99.50% | 98.47% | 98.47% | 0.04% | 0.00% | 0.04% |
| | | Physiological | 99.73% | 99.03% | 99.07% | 0.00% | 0.00% | 0.06% |
| | | saline | | | | | | |
| 48 | h | CE01KS | 99.36% | 99.41% | 98.49% | 0.00% | 0.00% | 0.12% |
| | | Physiological | 98.25% | 98.33% | 95.97% | 0.02% | 0.00% | 0.24% |
| | | saline | | | | | | |

Table 4 shows the results of surface markers at different time points in the refrigerator. It can be seen that the positive rate was greater than 95% and the negative rate was less than 2%.

### EXPERIMENTAL CONCLUSIONS

Maintaining good cell viability and cell functions represents the greatest challenge for clinically used cells to exert their efficacy. In this experiment, a compound electrolyte was added with an amino acid, a vitamin, as well as glucose and sodium chloride. The cells were dispensed according to the standard operating procedure of cell suspension preparation. The cells were preserved in a refrigerator at 2° C. to 8° C., and samples were taken at 0 h, 24 h, 48 h, and 72 h respectively. The stability of cell viability, the trend of change of cell apoptosis, the cell surface markers, the cell morphology, and the cell proliferation ability at different time points were compared, and the following results were obtained:

There was not much difference in cell morphology between the cells preserved with CE01KS and the cells preserved with physiological saline. However, for cell therapy the FDA has established the standard on minimal cell survivability (generally set at 70%). Cells preserved with CE01KS of the present invention for 72 h had a cell viability of greater than 70%, the apoptosis of the cells were very slow (in contrast, cells preserved with physiological saline could only sustain for 24 h), and the cell surface markers changed little after dispensing, with the positive rate being above 95% and the negative rate being below 2% for various time points.

In summary, CE01KS can preserve cells better than physiological saline.

All documents mentioned in the present invention are cited as references in the present application as if each document is cited separately as a reference. In addition, it should be understood that various alternations or modifications to the present invention can be made by a person skilled in the art after reading the foregoing disclosure of the present invention, and these equivalents also fall within the scope defined in the appended claims of the present application.

1. A cell preservation solution, characterized by comprising:
a compound electrolyte;
glucose, and sodium chloride;
a vitamin, and
an amino acid.

2. The cell preservation solution according to claim 1, wherein the vitamin is a water-soluble vitamin.

3. The cell preservation solution according to claim 1, wherein the volume of the amino acid accounts for 0.2% to 3% of the total volume of the preservation solution.

4. The cell preservation solution according to claim 1, comprising: 0.93-1.72 g/ml of sodium chloride, 0.86-1.44 g/ml of sodium gluconate, 0.64-1.44 g/ml of sodium acetate, 0.04-1.44 g/ml of potassium chloride, and 0.04-1.44 g/mL of magnesium chloride.

5. The cell preservation solution according to claim 1, comprising 0.32-1.8 g/ml of glucose.

6. A mixture of adipose-derived mesenchymal stem cells, characterized by comprising adipose-derived mesenchymal stem cells and the preservation solution according to any one of claims 1-5.

7. The mixture of adipose-derived mesenchymal stem cells according to claim 6, wherein the ratio of the adipose-derived mesenchymal stem cells to the preservation solution is 1-5×10⁷ cells: 1-3 mL preservation solution.

8. A method for cryopreservation of cells, characterized by comprising the steps of suspending cells in the cell preservation solution according to claim 1, and then preserving the cells at a low temperature.

9. A repository of adipose-derived mesenchymal stem cells, characterized by comprising the mixture of adipose-derived mesenchymal stem cells according to claim 6.

10. Use of the preservation solution according to any one of claims 1-5 in the preservation of adipose-derived mesenchymal stem cells, the establishment of a repository of adipose-derived mesenchymal stem cells, the maintenance of the viability of adipose-derived mesenchymal stem cells, or the retainment of surface markers of adipose-derived mesenchymal stem cells.
